(19) United States
(12) Patent Application Publication
(10) Pub. No.: US 2008/0139672 A1
(43) Pub. Date: Jun. 12, 2008

(54) SYNERGISTIC PRESERVATIVE SYSTEMS AND THEIR USE IN COSMETIC COMPOSITIONS

(75) Inventors: Ellen Rozsa, Bergenfield, NJ (US); Ovid Romanoschi, Bergenfield, NJ (US); Olga Dueva-Koganov, White Plains, NY (US)

Correspondence Address: OHLANDT, GREELEY, RUGGIERO & PERLE, LLP, ONE LANDMARK SQUARE, 10TH FLOOR, STAMFORD, CT, 06901, US

(73) Assignee: Playtex Products, Inc., Westport, CT (US)

(21) Appl. No.: 11/672,425

(22) Filed: Feb. 7, 2007

Related U.S. Application Data
(63) Continuation-in-part of application No. 11/353,567, filed on Feb. 14, 2006.

(51) Int. Cl. A61K 47/10; A61K 47/12; A61Q 99/00

(52) U.S. Cl. 514/784; 514/772

(57) ABSTRACT

The present invention provides a preservative system having at least one preservative component and at least one oil-miscible glycol component, and cosmetic compositions formulated with the preservative system. It has unexpectedly been found that the preservative system according to the present invention results in a synergistic preservative effect. This synergistic preservative effect allows for the use of a reduced amount of the preservative component when formulating a cosmetic composition, which in turn results in a composition with reduced irritation potential and reduced material costs.

### CROSS REFERENCE TO RELATED APPLICATIONS

This application is a continuation-in-part of pending U.S. patent application Ser. No. 11/353,567 filed Feb. 14, 2006.

### BACKGROUND OF THE INVENTION

1. Field of Invention

The present invention relates generally to preservative systems and their use in compositions. More particularly, the present invention relates to synergistic preservative systems and their use in cosmetic compositions.

2. Description of Related Art

Preservatives are frequently used in all types of cosmetic compositions to prevent decay and breakdown of such compositions mainly caused by microorganisms. Effective preservatives must have two types of activity, namely anti-microbial activity and anti-fungal activity. Most of the current preservatives contain some form of paraben, often in combination with phenols and quaternary compounds. Some of these paraben preservative systems, however, are limited in their global acceptability and are not allowed in either Europe or Japan.

Various parabens have shown estrogenic activity and other deleterious effects on reproductive systems in various test models. See Pedersen, K. L. et al., The preservatives ethyl-, propyl- and butylparaben are estrogenic in an in vivo fish assay, Pharmacology & Toxicology (Vol. 86(3), pp 110-13, March 2000); Routledge, E. J., et al., Some alkyl hydroxy benzoate preservatives (parabens) are estrogenic, Toxicology and Applied Pharmacology (Vol. 153(1), pp. 12-19 (November 1998); and Kang, K. S. et al, Decreased sperm number and motile activity on the F1 offspring maternally exposed to butyl p-hydroxybenzoic acid (butyl paraben), Journal of Veterinary Medical Science (Vol. 64(3), pp. 227-35 (March 2002); and Philippa Darbre and Philip Harvey, Endocrine disrupters and human health: could estrogenic chemicals in body care cosmetics adversely affect breast cancer incidence in women?, Journal of Applied Toxicology, 2004—June; 24 (3): 167-76.

Likewise, while effective, other preservative systems, such as, formaldehyde donors, isothiazolinones, and ethanols are defective in numerous safety/compatibility-related issues, for example, high irritation potential and incompatibility with avobenzone. Therefore, glycol, by itself, cannot substitute the anti-bacterial and anti-fungal properties of a preservative in a typical cosmetic composition.

It is also known that certain glycols offer some anti-bacterial efficacy at relatively high concentrations of glycol, in the range between 1% to 7%, based on a total weight of a composition. In addition, it was also found that glycols have limited or no anti-fungal efficacy,

Accordingly, while there have been attempts in the prior art to formulate paraben-free compositions, there is still a strong need in the art for a preservative composition or system that is not only paraben-free, but also has less irritation potential, has both antimicrobial and fungicidal activity, and has lower manufacturing costs. The present invention meets these needs.

### SUMMARY OF THE INVENTION

It is an object of the present invention to provide a preservative system suitable for use in cosmetic compositions.

It is another object of the present invention to provide such a preservative system that includes at least one preservative component and at least one oil-miscible glycol component.

It is still another object of the present invention to provide such a preservative system that exhibits synergistic properties.

It is a further object of the present invention to provide such a preservative system that provides both antimicrobial and fungicidal activities.

It is still a further object of the present invention to provide such a preservative system that reduces the required amounts of preservative component while maintaining adequate preservative efficacy in a cosmetic composition, which leads to cost reductions and lower irritation potential.

It is yet a further object of the present invention to provide such a preservative system with increased antioxidant and moisturizing potential.

It is another object of the present invention to provide improved ease of formulation of a cosmetic composition with respect to color, pH, and viscosity.

These and other objects of the present invention are achieved by providing a preservative system having at least one preservative component and at least one oil-miscible glycol component, and cosmetic compositions formulated with the preservative system. It has unexpectedly been found that the preservative system according to the present invention results in a synergistic preservative effect. This synergistic preservative effect allows for the use of a reduced amount of the preservative component when formulating a cosmetic composition, which in turn results in a composition with reduced irritation potential and reduced material costs.

### DETAILED DESCRIPTION OF THE INVENTION

The present invention provides enhanced or synergistic preservative systems for use in topical compositions, such as cosmetic and dermatological compositions. In particular, it has been found that a preservative system with one or more oil-miscible glycols and one or more preservative components results in a synergistic preservative system that is effective for use in topical compositions. These synergistic preservative systems demonstrate adequate anti-microbial efficacy without the use of harsher preservative systems, such as, formaldehyde donors, parabens, ethanols, and isothiazolinones. In one embodiment of the present invention, the preservative system is free of formaldehyde donors, parabens, ethanols, and isothiazolinones. These systems may support preservative-free product claims and may also provide anti-oxidant and moisturization benefits.

While some glycols have anti-bacterial efficacy, their efficacy is proficient only at relatively high concentrations. When one or more oil-miscible glycols are combined with one or more preservative components according to the present invention, however, the combination unexpectedly results in a synergistic preservative system that is free of harsh preservatives, but is still anti-fungal and/or anti-bacterial. Furthermore, as a result of the synergism between the one or more oil-miscible glycols and the one or more preservative components, reduced concentrations of each are required in a cosmetic composition to achieve anti-fungal and/or anti-bacterial activity in the present invention.

Additionally, the use of the one or more oil-miscible glycols in the present invention provides broader formulation opportunities. This includes, but is not limited to, allowing for formulating systems and/or compositions with low water content.

Suitable oil-miscible glycols for use in the present invention include, but are not limited to, one or more of pentylene glycol, neopentyl glycol, caprylyl glycol, benzyl glycol, hexanediol, ethyl hexanediol, or any combinations thereof. In one embodiment according to the present invention, the glycol is caprylyl glycol, which is, by way of example, commercially available from Inolex Chemical Company under the trade name LexGard® O. In another embodiment, the glycol is a neopentyl glycol, which is, by way of example, commercially available from Inolex Chemical Company under the tradename LexFeel® 7.

The one or more oil-miscible glycols are present in the preservative system in an amount about 50 wt. % to about 90 wt. % based on the total weight of the preservative system. In one embodiment, the one or more oil-miscible glycols are present in an amount about 65 wt. % to about 85 wt. %, based on the total weight of the preservative system. In yet another embodiment, the one or more oil-miscible glycols are present in an amount about 75 wt. % to about 85 wt. %, based on the total weight of the preservative system.

The one or more oil-miscible glycols are present in a cosmetic composition in an amount about 0.1 wt. % to about 5 wt. %, based on the total weight of the cosmetic composition. In one embodiment of the present invention, the one or more oil-miscible glycols are present in an amount about 0.5 wt. % to about 4 wt. %, based on the total weight of the cosmetic composition. In another embodiment of the present invention, the one or more oil-miscible glycols are present in an amount about 0.75 wt. % to about 3.25 wt. %, based on the total weight of the cosmetic composition.

Suitable preservative components for the use in the preservative system according to the present invention include, but are not limited to, at least one organic acid, organic acid derivative, inorganic acid, inorganic acid derivative, or any combinations thereof.

Suitable organic acid or organic acid derivative preservative component for use in the preservative system of the present invention include, but are not limited to, erythrobic acid, benzoic acid, citric acid, sorbic acid, glucono-1,5-lactone (GDL), which is a neutral cyclic ester of gluconic acid, lactic acid, calcium lactate, sodium erythorbate, sodium benzoate, sodium citrate, sodium sorbate, potassium sorbate, sodium gluconate, EDTA, disodium EDTA, trisodium EDTA, tetrasodium EDTA, or any combinations thereof.

Suitable inorganic acid or derivatives for the preservative components for use in the present invention include, but are not limited to, bicarbonate, sodium bicarbonate, potassium bicarbonate, sodium sulfite, sodium metabisulfite, or any combinations thereof.

In one embodiment of the present invention, the preservative component of the preservative system includes a combination of organic acid or derivative and inorganic acid or derivative. In this embodiment, the components are present in a ratio of organic acid or derivative to inorganic acid or derivative between about 1:1 to about 4:1. In another embodiment, they are present in a ratio of organic acid or derivative to inorganic acid or derivative between about 1:1 to about 1:4. In another embodiment, they are present in a ration of organic acid or derivative to inorganic acid or derivation between about 1:4 to about 4:1.

In one embodiment of the present invention, the preservative component of the preservative system includes a combination of organic acid or derivative and inorganic acid or derivative. In this embodiment, the components are present in a ratio of inorganic acid or derivative to inorganic acid or derivative between about 1:1 to about 4:1. In another embodiment, they are present in a ratio of organic acid or derivative to inorganic acid or derivative between about 1:1 to about 1:4. In another embodiment, they are present in a ratio of organic acid or derivative to inorganic acid or derivation between about 1:4 to about 4:1.

In one embodiment of the present invention, the preservative system includes one or more of GDL, sodium erythorbate, sodium benzoate, disodium EDTA, or any combinations thereof.

The one or more preservative components are present in the preservative system in an amount about 10 wt. % to about 50 wt. %, based on the total weight of the preservative system. In one embodiment, the one or more preservative components are present in an amount about 15 wt. % to about 35 wt. %, based on the total weight of the preservative system. In another embodiment of the present invention, the one or more preservative components are present in an amount about 15 wt. % to about 25 wt. %, based on the total weight of the preservative system.

The one or more preservative components are present in a final cosmetic composition in an amount about 0.05 wt. % to about 5 wt. %, based on the total weight of the cosmetic composition. In one embodiment, the one or more preservative components are present in an amount about 0.075 wt. % to about 2.5 wt. %, based on the total weight of the cosmetic composition. In another embodiment of the present invention, the one or more preservative components are present in an about 0.1 wt. % to about 0.75 wt. %, based on the total weight of the cosmetic composition.

By way of example, in another embodiment of the present invention, suitable preservative components for use in the present invention include the Natrulon GPS family of components sold commercially by Lonza. The Cosmetic, Toiletry, and Fragrance Association (CTFA) list selected components of NATRULON GPS as antioxidants/moisturizers. Natrulon GPS # 4 includes 75% glucono-delta-lactone (GDL) and 25% sodium erythorbate. Natrulon GPS # 5 includes 75% GDL and 25% sodium benzoate. Natrulon GPS # 6 includes 60% GDL, 20% sodium erythorbate and 20% sodium sulfite. Also, oil-miscible glycols are known to have humectant properties. Therefore, in addition to providing enhanced anti-microbial and fungicidal properties to cosmetic compositions, the synergistic preservative system of the present invention may also impart desirable antioxidant and moisturizing properties to these compositions.

In one embodiment of the present invention, the preservative system used in a cosmetic composition includes one or more oil-miscible glycols and one or more of the Natrulon GPS components. The one or more oil-miscible glycols are present in an amount about 0.5 wt. % to about 3.5 wt. %, based on the total weight of the cosmetic composition. The one or more Natrulon GPS components are present in an amount about 0.45 wt. % to about 0.65 wt. %, based on the total weight of the cosmetic composition. These embodiments of the present invention yield surprisingly positive results despite the reduction in the amount of both the glycol and preservative components compared to the amounts typically used and/or recommended for use in cosmetic compositions. It was found that when used in a cosmetic composition, such combinations, in such amounts, provide both anti-microbial and anti-fungicidal activities.

The cosmetic composition formulated with the synergistic preservative system according to the present invention may also include one or more additional components or ingredients including, but not limited to, solvents, surfactants,-emulsifiers, emollients, humectants, moisturizers, thickeners, antioxidants, vitamins, sunscreen agents, additional preservatives, pH adjusters, chelating agents, viscosity modifiers, or any combinations thereof.

The present invention also relates to a cosmetic composition formulated with the synergistic preservative system of the present invention. The cosmetic composition may be any known cosmetic composition, such as, for example, sunscreen, personal care, skin care, hair care, color cosmetic, anti-aging, or any combinations thereof. The cosmetic compositions may be in any suitable form, including, but not limited to, lotion, cream, spray, gel, foam, powder, stick, or any combinations thereof.

The synergistic preservative system of the present invention may be included in a cosmetic composition in an amount about 0.15 wt. % to about 10 wt. %, based on the total weight of the cosmetic composition. In one embodiment of the present invention, the synergistic preservative system is present in a cosmetic composition in an amount about 0.575 wt. % to about 6.5 wt. %, based on the total weight of the cosmetic composition. In another embodiment of the present invention, the synergistic preservative system is present in a cosmetic composition in an amount about 0.85 wt. % to about 4 wt. %, based on the total weight of the cosmetic composition.

The following example is merely illustrative of a cosmetic composition formulated with a synergistic preservative system according to the present invention. The example is not intended to limit the scope of the invention.

### EXAMPLE

Referring to Table 1 below, Formula A represents a cosmetic composition without a synergistic preservative system according to the present invention. Formulas B and C represent cosmetic compositions formulated with a synergistic preservative system according to the present invention.

TABLE 1

| | | TOTAL WT % | TOTAL WT % | TOTAL WT % |
|---|---|---|---|---|
| CHEMICAL/INCI/USP NAME | TRADE NAME | A | B | C |
| Purified Water | | 65.10 | 64.10 | 64.10 |
| Octyl Salicylate | Neo Heliopan OS | 5.00 | 5.00 | 5.00 |
| Homosalate | Escalol 567 | 7.50 | 7.50 | 7.50 |
| Avobenzone | Parsol 1789 | 3.00 | 3.00 | 3.00 |
| Octocrylene | | 2.50 | 2.50 | 2.50 |
| Oxybenzone | | 1.50 | 1.50 | 1.50 |
| Cetearyl Alcohol (and) Dicetyl Phosphate | Crodophos CES | 5.50 | 5.50 | 5.50 |
| (and) Ceteth-10 Phosphate | | | | |
| Polyacrylamidomethylpropane Sulfonic | Cosmedia Polymer | 4.50 | 5.00 | 5.00 |
| Acid | HSP 1180 | | | |
| Caprylyl Glycol | LexGard O | 3.50 | 2.00 | 2.00 |
| Sodium Hydroxide, 10% soln | | 1.80 | 3.10 | 3.10 |
| Disodium EDTA | Dissolvine Na2 | 0.10 | 0.10 | 0.10 |
| Xanthan Gum | | 0.00 | 0.20 | 0.20 |
| Glucono Delta Lactone (GDL) (and) | Natrulon GPS # 4 | | 0.50 | |
| Sodium Erythrobate* | | | | |
| Glucono Delta Lactone (GDL) (and) | Natrulon GPS# 5 | | | 0.50 |
| Sodium Benzoate** | | | | |
| | | 100.00 | 100.00 | 100.00 |

*75% Glucono Delta Lactone (GDL) (and) 25% Sodium Erythrobate
**75% Glucono Delta Lactone (GDL) (and) 25% Sodium Benzoate

In the Table 2 below, Formulas D and E represent additional examples of a cosmetic compositions formulated with a synergistic preservative systems according to the present invention.

TABLE 2

| | | TOTAL WT | TOTAL WT |
|---|---|---|---|
| | | % | % |
| CHEMICAL/INCI/USP NAME | TRADE NAME | D | E |
| Purified Water, USP | Purified Water, USP | 61.556 | 61.556 |
| Mineral Oil and Lanolin Alcohol | Amerchol L-101 | 1.440 | 1.440 |
| Lanolin, Anhydrous | Cosmetic Lanolin (Rita) | 0.144 | 0.144 |
| Glyceryl Stearate | Witconol MST | 2.100 | 2.100 |
| Glyceryl Stearate and PEG-100 Stearate | Ariacel 165 | 2.750 | 2.750 |
| Cetyl Alcohol | Adol 52 NF | 1.600 | 1.600 |
| Octinoxate | | 7.000 | 7.000 |
| Octocrylene | | 1.250 | 1.250 |
| Octisalate | | 5.000 | 5.000 |
| Avobenzone | Parsol 1789 | 1.500 | 1.500 |
| Neopentyl Glycol Diheptanoate | LexFeel 7 | 2.500 | 2.500 |
| Caprylyl Glycol | LexGard O | 0.750 | 0.750 |
| Tocopherol | Tocopherol USP-FCC | 0.010 | 0.010 |
| Retinyl Palmitate | Retinyl Palmitate Type P1.7E | 0.010 | 0.010 |
| Cholecalciferol | Liquid Vitamin D3 in corn oil | 0.010 | 0.010 |
| Dimethicone | Silicone Fluid 350 cst | 1.000 | 1.000 |
| Xanthan Gum | Vanzan NF | 0.350 | 0.350 |
| Disodium EDTA | Dissolvine Na2X | 0.100 | 0.100 |
| Glycerin, USP | Glycon G-100 | 6.000 | 6.000 |
| Sodium Hydroxide 10% solution qs to pH~6.0 | | 3.520 | 3.520 |
| Allantoin | | 0.500 | 0.500 |
| Aloe Vera Gel | Aloe-Con UP-40 | 0.010 | 0.010 |
| Glucono Delta Lactone (GDL) (and) Sodium | Natrulon GPS # 4 | | 0.600 |
| Erythrobate* | | | |
| Glucono Delta Lactone (GDL) (and) Sodium | Natrulon GPS# 5 | 0.600 | |
| Benzoate** | | | |
| Fragrance | | 0.300 | 0.300 |
| Total | | 100.000 | 100.000 |

*75% Glucono Delta Lactone (GDL) (and) 25% Sodium Erythrobate
**75% Glucono Delta Lactone (GDL) (and) 25% Sodium Benzoate

Formulas A through E were tested with the following bacteria and fungi for in vitro effectiveness:

Gram negative bacilli for bacterial pool:

| | |
|---|---|
| A. Pseudomonas aeruginosa | ATCC 9027 |
| B. Burkholderia cepacia | ATCC 25416 |

Gram positive cocci for bacterial pool:

| | |
|---|---|
| A. Staphylococcus aureus | ATCC 6538 |

Enteric organisms for bacterial pool:

| | |
|---|---|
| A. Eschericia coli | ATCC 8739 |
| B. Klebsiella pneumoniae | ATCC 13883 |

Fungi pool:

| | |
|---|---|
| A. Candida albicans | ATCC 10231 |
| B. Penicillium chrysogenum | ATCC 10106 |
| C. Aspergillus niger | ATCC 16404 |

The working cultures of the bacteria and fungi were prepared using the ATCC Culti-Loop (Bio-Mereiux-Remel). Bacterial and fungi cultures were inoculated and incubated to produce the desired inoculum pools that are appropriate for the testing.

Each inoculum pool was then mixed with a 50 gram sample of test product and was shaken vigorously to create a homogeneous mixture. Each mixture then was stored at ambient temperature and humidity. Water miscible products in the mixture were further treated with a modified Williamson Buffer Solution (WBS) (0.4 g of KH₂PO₄, 10.1 g of NA2HPO₄, 10.0 mL of Tween 80, 0.09 g of sodium bisulfite in 1 L of DI water) to render homogeneous and dispersed. 1.0 mL of the 1:10 dilution in WBS of each mixture was dispensed into its respective labeled petri dish with 20 mL of Trypticase Soy Broth for each bacterial plate and 20 mL of Sabouraud Dextrose Agar for each fungi plate. The plates were allowed to solidify and were incubated and re-inoculated after 7 days. The dishes were assayed at 7, 14, and 21 days after the re-inoculation for a total test period of 28 days.

Product is considered to be adequately preserved (passed) if the bacterial count is <10 colony forming units (CFU)/gm at day 7 and day 14, post inoculation, with no increase in growth for the remaining test period. Product is adequately preserved (passed) if the fungal count is <1000 CFU/gm at day 7 and day 14, post inoculation, with no increase in growth for the remaining test period.

The results presented in Table 3 indicate that that the use of caprylyl glycol alone even at high concentration, 3.5%, failed to provide adequate preservative properties (Formula A). In contrast, caprylyl glycol in combination with glucono-delta-lactone and sodium erythorbate and sodium benzoate provides proficient preservative properties (Formulas B and E with sodium erythorbate; and Formulas C and D with sodium benzoate). Total concentrations of compositions of glucono-delta-lactone and sodium erythorbate and glucono-delta-lactone and sodium benzoate were in the range of from 0.5% to 0.6%—below their effective level. The effective concentrations of caprylyl glycol were in the range of 0.75-2%.

TABLE 3
The results of the determination of in vitro efficacy of anti-
microbial agents in Formulations A through E:

| | | | | | | |
|---|---|---|---|---|---|---|
| | | Day 7: | | | | |
| Day 1 | Day 3 | Reinoculation | Day 14 | Day 21 | Day 28 | Result |

Composition A

| | | | | | | | |
|---|---|---|---|---|---|---|---|
| Bacilli, CFU/gm | 0 | 0 | 0 | 0 | 20 | 10 | Failed |
| Fungi, CFU/gm | 200 | 300 | 100 | 300 | 400 | 400 | Failed |

Composition B

| | | | | | | | |
|---|---|---|---|---|---|---|---|
| Bacilli, CFU/gm | 0 | 0 | 0 | 0 | 0 | 0 | Passed |
| Fungi, CFU/gm | 40 | 0 | 0 | 0 | 0 | 0 | Passed |

Composition C

| | | | | | | | |
|---|---|---|---|---|---|---|---|
| Bacilli, CFU/gm | 0 | 0 | 0 | 0 | 0 | 0 | Passed |
| Fungi, CFU/gm | 10 | 0 | 0 | 0 | 0 | 0 | Passed |

Composition D

| | | | | | | | |
|---|---|---|---|---|---|---|---|
| Bacilli, CFU/gm | 0 | 0 | 0 | 0 | 0 | 0 | Passed |
| Fungi, CFU/gm | 20 | 0 | 0 | 0 | 0 | 0 | Passed |

Composition E

| | | | | | | | |
|---|---|---|---|---|---|---|---|
| Bacilli, CFU/gm | 0 | 0 | 0 | 0 | 0 | 0 | Passed |
| Fungi, CFU/gm | 60 | 40 | 0 | 0 | 0 | 0 | Passed |

The present invention has been described with particular reference to the preferred forms thereof. It will be obvious to one of ordinary skill in the art that changes and modifications may be made therein without departing from the spirit and scope of the present invention as set forth above and the claims below.

What is claimed is:

1. A synergistic preservative system for use in a cosmetic composition comprising:
one or more oil-miscible glycols; and
one or more preservative components,
wherein said synergistic preservative system demonstrates enhanced anti-fungal and/or anti-bacterial efficacy and is free of formaldehyde donors, parabens, ethanols, and isothiazolinones.

2. The synergistic preservative system of claim 1, wherein said one or more oil-miscible glycols is selected from the group consisting of pentylene glycol, neopentyl glycol, caprylyl glycol, benzyl glycol, hexanediol, ethyl hexanediol, and any combinations thereof.

3. The synergistic preservative system of claim 1, wherein said one or more oil-miscible glycols are present in said preservative system in an amount about 50 wt. % to about 90 wt. % based on the total weight of the preservative system.

4. The synergistic preservative system of claim 1, wherein said one or more oil-miscible glycols are present in said preservative system in an amount about 75 wt. % to about 85 wt. %, based on the total weight of the preservative system.

5. The synergistic preservative system of claim 1, wherein said one or more preservative components are selected from the group consisting of organic acid, organic acid derivative, inorganic acid, inorganic acid derivative, and any combinations thereof.

6. The synergistic preservative system of claim 5, wherein said organic acid or said organic acid derivative is selected from the group consisting of erythrobic acid, benzoic acid, citric acid, sorbic acid, gluconic acid, glucono-1,5-lactone (GDL), lactic acid, calcium lactate, sodium erythorbate, sodium benzoate, sodium citrate, sodium sorbate, potassium sorbate, sodium gluconate, EDTA, disodium EDTA, trisodium EDTA, tetrasodium EDTA, and any combinations thereof.

7. The synergistic preservative system of claim 5, wherein said inorganic acid and said inorganic acid derivatives are selected from the group consisting of bicarbonate, sodium bicarbonate, potassium bicarbonate, sodium sulfite, sodium metabisulfite, and any combinations thereof.

8. The synergistic preservative system of claim 1, wherein said one or more preservative components are selected from the group consisting of GDL, sodium erythorbate, sodium benzoate, disodium EDTA, and any combinations thereof.

9. The synergistic preservative system of claim 1, wherein said one or more preservative components are present in said preservative system in an amount about 10 wt. % to about 50 wt. %, based on the total weight of the preservative system.

10. The synergistic preservative system of claim 1, wherein said one or more preservative components are present in said preservative system in an amount about 15 wt. % to about 25 wt. %, based on the total weight of the preservative system.

11. The synergistic preservative system of claim 1, wherein said one or more preservative components are a combination of at least one organic acid or derivative and at least one inorganic acid or derivative.

12. The synergistic preservative system of claim 11, wherein said at least one organic acid or derivative and said at least one inorganic acid or derivative are present in a ratio of said at least one organic acid or derivative to said at least one inorganic acid or derivative between about 1:4 to about 4:1.

13. A synergistic preservative system for use in a cosmetic composition comprising:
one or more oil-miscible glycols selected from the group consisting of pentylene glycol, neopentyl glycol, caprylyl glycol, benzyl glycol, hexanediol, ethyl hexanediol, and any combinations thereof; and
one or more preservative components selected from the group consisting of glucono-delta-lactone, sodium erythorbate, sodium benzoate, sodium sulfite, and any combinations thereof.

14. The synergistic preservative system of claim 13, wherein said synergistic preservative system is free of formaldehyde donors, parabens, ethanols, and isothiazolinones.

15. The synergistic preservative system of claim 13, wherein said one or more oil-miscible glycols are present in said preservative system in an amount about 50 wt. % to about 90 wt. % based on the total weight of the preservative system.

16. The synergistic preservative system of claim 13, wherein said one or more oil-miscible glycols are present in an amount about 75 wt. % to about 85 wt. %, based on the total weight of the preservative system.

17. The synergistic preservative system of claim 13, wherein said one or more preservative components are present in said preservative system in an amount about 10 wt. % to about 50 wt. %, based on the total weight of the preservative system.

18. The synergistic preservative system of claim 13, wherein said one or more preservative components are present in said preservative system in an amount about 15 wt. % to about 25 wt. %, based on the total weight of the preservative system.

19. A cosmetic composition comprising a synergistic preservative system comprising:
one or more oil-miscible glycols; and
one or more preservative components,
wherein said cosmetic composition is free of formaldehyde donors, parabens, ethanols, and isothiazolinones.

20. The cosmetic composition of claim 19, wherein said one or more oil-miscible glycols is selected from the group consisting of pentylene glycol, neopentyl glycol, caprylyl glycol, benzyl glycol, hexanediol, ethyl hexanediol, and any combinations thereof.

21. The cosmetic composition of claim 19, wherein said one or more oil-miscible glycols are present in said cosmetic composition in an amount about 0.1 wt. % to about 5 wt. %, based on the total weight of the cosmetic composition.

22. The cosmetic composition of claim 19, wherein said one or more oil-miscible glycols are present in said cosmetic composition in an amount about 0.75 wt. % to about 3.25 wt. %, based on the total weight of the cosmetic composition.

23. The cosmetic composition of claim 19, wherein said one or more preservative components are selected from the group consisting of organic acid, organic acid derivative, inorganic acid, inorganic acid derivative, and any combinations thereof.

24. The cosmetic composition of claim 23, wherein said organic acid or said organic acid derivative is selected from the group consisting of erythrobic acid, benzoic acid, citric acid, sorbic acid, gluconic acid, glucono-1,5-lactone (GDL), sodium erythorbate, lactic acid, calcium lactate, sodium benzoate, sodium citrate, sodium sorbate, potassium sorbate, sodium gluconate, EDTA, disodium EDTA, trisodium EDTA, tetrasodium EDTA, and any combinations thereof.

25. The cosmetic composition of claim 23, wherein said inorganic acid and said inorganic acid derivatives are selected from the group consisting of bicarbonate, sodium bicarbonate, potassium bicarbonate, sodium sulfite, sodium metabisulfite, and any combinations thereof.

26. The cosmetic composition of claim 19, wherein said one or more preservative components are selected from the group consisting of GDL, sodium erythorbate, sodium benzoate, disodium EDTA, and any combinations thereof.

27. The cosmetic composition of claim 19, wherein said one or more preservative components are present in said cosmetic composition in an amount about 0.05 wt. % to about 5 wt. %, based on the total weight of the cosmetic composition.

28. The cosmetic composition of claim 19, wherein said one or more preservative components are present in said cosmetic composition in an amount about 0.1 wt. % to about 0.75 wt. %, based on the total weight of the cosmetic composition.

29. The cosmetic composition of claim 19, wherein said preservative system is present in said cosmetic composition in an amount about 0.15 wt. % to about 10 wt. %, based on the total weight of the cosmetic composition.

30. The cosmetic composition of claim 19, wherein said preservative system is present in said cosmetic composition in an amount about 0.85 wt. % to about 4 wt. %, based on the total weight of the cosmetic composition.

31. The cosmetic composition of claim 19, wherein said one or more preservative components are a combination of at least one organic acid or derivative and at least one inorganic acid or derivative.

32. The cosmetic composition of claim 31, wherein said at least one organic acid or derivative and said at least one inorganic acid or derivative are present in a ratio of said at least one organic acid or derivative to said at least one inorganic acid or derivative between about 1:4 to about 4:1.

33. The cosmetic composition of claim 19, wherein said cosmetic composition is a composition selected from the group consisting of sunscreen, personal care, skin care, hair care, color cosmetic, anti-aging, and any combination thereof.

34. The cosmetic composition of claim 19, wherein said cosmetic composition is in a form selected from the group consisting of lotion, cream, spray, gel, foam, powder, stick, and any combinations thereof.

35. The cosmetic composition of claim 19, further comprising one or more additional components selected from the group consisting of solvents, surfactants, emulsifiers, emollients, humectants, moisturizers, thickeners, antioxidants, vitamins, sunscreen agents, additional preservatives, pH adjusters, chelating agents, viscosity modifiers, and any combinations thereof.

36. The cosmetic composition of claim 19, further comprising a bacterial count that is less than 10 colony forming units per gram at day 7 post inoculation and a fungal count that is less than 1000 colony forming units per gram at day 7 post inoculation.

37. The cosmetic composition of claim 19, further comprising a bacterial count that is less than 10 colony forming units per gram at day 14 post inoculation and a fungal count that is less than 1000 colony forming units per gram at day 14 post inoculation.
